(19) United States
(12) Patent Application Publication
(10) Pub. No.: US 2016/0073614 A1
(43) Pub. Date: Mar. 17, 2016

(54) System and Method for Detection of Lameness in Sport Horses and other Quadrupeds

(71) Applicants: Kyle Douglas Lampe, Bellevue, WA (US); Rachel Lampe, Bellevue, WA (US)

(72) Inventors: Kyle Douglas Lampe, Bellevue, WA (US); Rachel Lampe, Bellevue, WA (US)

(21) Appl. No.: 14/732,880

(22) Filed: Jun. 8, 2015

Related U.S. Application Data
(63) Continuation-in-part of application No. 14/486,540, filed on Sep. 15, 2014, now abandoned.

(51) Int. Cl. A01K 29/00; A61B 5/00; A61B 5/11

(52) U.S. Cl. CPC A01K 29/005; A61B 5/112; A61B 5/1127; A61B 5/0075; A61B 5/0077; A61B 5/7267; A61B 5/7282; A61B 5/0013; A61B 5/6893; A61B 2503/40; A61B 2576/00

(57) ABSTRACT

A method of diagnosing lameness in quadrupeds utilizing computer vision and a computerized depth perception system to scan quadrupeds, such as sport horses, over time. The method enables a detailed analysis of the quadruped's movement, and changes thereof over time without the need for attaching sensors to the body of the horse, or requiring force plates or expensive high speed cameras. A processing system receives the input of this movement data and utilizes it to make a determination of severity of lameness signals of the animal. The system is inexpensive enough that non-specialists, such as non-veterinary trained quadruped owners, may install the system at an appropriate location such as a horse barn enabling identification of lameness early, to aid in objectively analyzing rehabilitation from injury, and relating changes in gait to performance changes.

### CROSS-REFERENCE TO RELATED APPLICATIONS

The present application is a continuation in part application which claims the benefit of U.S. Nonprovisional patent application Ser. No. 14/486,540 filed Sep. 15, 2014 which claims the benefit of U.S. Provisional Patent Application No. 61/877,694 filed Sep. 13, 2013, entitled “System for Detecting Lameness in Sport Horses and other Quadrupeds,” and which are both incorporated herein by reference in their entirety as if fully set forth herein.

### FIELD OF THE DISCLOSURE

This disclosure relates generally to the field of methods and systems for detecting lameness is quadrupeds, and more specifically, to detection of lameness in sport horses and other quadrupeds.

### BACKGROUND

Species of four-legged animals (quadrupeds), have a unique and healthy way of moving or ambulating (referred to as its “gait”). The gait of a quadruped is generally recognizable and consistent within a species by analyzing several biomechanical signals. Highly skilled and trained veterinarians are typically used to determine if an animal has an unhealthy gait caused by injury, referred to as ‘lame’. However, detection of lameness in quadrupeds, namely horses and more specifically sport horses, is a significant and often subjective task for veterinarians (vets) and horse owners. Current methods used in clinical practice for detecting lameness in a horse are subjective, and requires a highly skilled veterinarian (vet). Studies have shown that even between vets there is a certain amount of subjectivity and question about the common practices involved in detecting lameness and performing a lameness exam. The studies have shown that each vet has their own way of detection and examination. Subjectivity and lack of common practice has led owners to seek multiple vet opinions for examination before taking action. This delay in detecting an injury in a sport horse will delay treatment, cost more, and possibly hinder the horse's future success.

A veterinary exam and lameness workup is currently the most common technique for evaluating lameness in horses. A lameness workup typically includes the vet watching the horse move at a walk and a trot (symmetrical gaits) across hard and soft surfaces, in a straight line and in a circle. The symmetry of the trot allows the vet to try and visually determine asymmetry of movement in the horse to pinpoint lameness. However, such diagnosis are prone to subjectivity, and when the lameness is subtle, studies have shown disagreement between vets.

Automation is a recent area of research with regard to lameness diagnostics. The goal of automation is to seek a more objective examination. The most common automation method used involves attaching many sensors (ranging from just a few to a hundred) to the horse, then filming the horse trotting with high speed cameras and tracking the sensor's movement. Currently the most accurate digital detection apparatus for automated lameness evaluation involves the animals trotting across force plates to measure how they distribute their weight amongst limbs. Force plates are weight measuring plates embedded in the ground which can digitally detect the weight of each point of contact on them. If a quadruped can be shown to not be putting more weight on one side leg than the other, lameness can be detected. However both methods require expensive custom setups which require the horse to be brought to the diagnosis facility and are incapable of remotely diagnosing a horse. They also require placing a harness and special sensors on the horse making it a less natural movement. Other methods of diagnosing lameness include attaching accelerometers on the horse and measuring difference in acceleration of different parts of the horse's body. Finally, custom horse shoes that can detect weight have been proposed as well, but all attempts have been too expensive and been deemed too time intensive to re-shoe the horse any time a lameness examination is required.

In order to detect an injury or lameness in a quadruped, the animal must be moving. No research has shown that injury to a leg can be shown while an animal is standing, while extensive research has shown differences in the ways quadrupeds walk and trot. Often pain will only occur due to movement and quadrupeds will compensate for this by changing how weight is distributed between their legs during these acts, compensating for the distribution of weight with their other body parts such as a head bob (a more pronounced upward movement of the quadruped's head during movement of a lame leg), changing the stride length, and changing the frequency and timing of the movement by individual legs.

Lameness can present itself in very subtle ways and thus precise measurements of the relative body parts (legs, head, haunches, among others) is essential. Greater accuracy can yield early detection and different degrees of lameness.

It is important to conduct detection of lameness when a quadruped moving naturally. Quadrupeds and especially sport horses have a unique speed of each of their gaits which can vary between animals. For example, two different sport horses may begin to trot at different forward speeds depending on many factors. Treadmills are often used as part of lameness detection because a horse can be forced to be in one place for an extended period time. However, it is impossible to make a treadmill exactly match a quadruped's natural speed for their gait. As a result, the quadruped may make changes to its gait which can interfere with automated lameness detection. Force plates can allow for detecting natural movement, but they suffer from the fact that only a few strides can be detected because of the long stride length of some quadrupeds.

All methods of lameness detection, both manual and automated, aim to be noninvasive. Invasive lameness detection methods, such as X-Rays and MRI's require sedation of an animal, which is often a difficult and expensive procedure. Further, anesthesia puts the health of the animal at risk. The animal's weight is also a factor, as it is difficult to get an unconscious 2000 pound animal into an MRI.

Current methods of automated lameness detection that require an expensive or time intensive setup and thus are not suited for regular use over an animal's lifetime and are only used at times when the quadruped is unhealthy. A system that was cheaper and did not require extensive setup on a horse could be used when a quadruped is healthy as well. Typically, a sport animal is only brought to see a trained veterinarian when an owner notices something is felt by a rider or visible to their eye in the movement of a quadruped. A system that is used regularly could pick up changes in the gait of the animal imperceptible to a typical owner and alert a veterinarian at an earlier stage. It would have the additional benefit of being able to identify healthy traits in an individual animal, such as sidedness of horses that can cause misdiagnosis of lameness.

For the forgoing reasons, there is a need for an automated system which can noninvasively examine a quadruped in motion using accurate measurements taken from the natural movements in order to timely diagnose and treat chronic and acute injuries.

### SUMMARY

The present invention is directed to a system and method that satisfies this need. The system comprises an apparatus which is able to timely, accurately and automatically measure data from a quadruped while in natural motion without the need for any external attachments to the quadruped. The system also comprises a method which utilizes data measured from the system to provide a visual output of the measurements in order to diagnose lameness and other injuries to the quadruped.

The system and apparatus will measure the movement of a quadruped animal through the combination of visual images and depth measurements (point clouds) taken over time. The system will utilize one or more cameras capable of capturing a series of images in rapid succession, in both human visible color space and infrared color space, of the quadruped moving. The system will also utilize one or more depth cameras which will take a series of depth measurements (point clouds). The system will utilize the combination of the two sets of data over time to measure movement and position of a quadruped's body, thus measuring gait.

By utilizing an accurate depth camera and a high resolution camera combined with infrared images of the quadruped, an accurate representation of the physical positions, velocities, and accelerations of the quadruped's body and body parts can be ascertained.

As the system does not attach any apparatus to the quadruped being diagnosed and allows natural movement of the quadruped at its preferred gait and is noninvasive to the animal.

As the system does not require any surgery or introduction of foreign bodies into the animal, it is noninvasive.

Furthermore, this system, when installed in a horse's own barn measures those horses over time, even at times when they are healthy. The system measures a baseline that will further help with the lameness diagnosis and rehabilitation. This is an improvement over existing automated lameness detection systems, as typically veterinarians only examine these horses when they are already lame. Compilations of repeated observations of individual animals, and the posture or altering posture thereof, over extended periods of time (longitudinal studies) of the quadruped movement, may allow earlier and more fine-tuned diagnosis of problems. It may also be used for rehabilitation to quantify when the horse has returned to its healthy movement.

In some embodiments, the System for Detecting Lameness in Sport Horses and other Quadrupeds is made up of a computer vision system operable to capture and record images and video, infrared images and video, and depth measurements of the quadruped. It is also comprised of a control system being operable to at least: receive an input from the computer vision system; receive input from the depth perception system; generate from the input three-dimensional representations of the specimen quadruped, wherein these representations are captured and recorded over time; utilize this series of three-dimensional representations for the creation of a mathematical representation of a movement of the specimen quadruped; utilize the computer vision system to identify an individual animal; update the mathematical representation of a movement of the specimen quadruped and detect deviations of the representation of the movement over time, either compared to a healthy baseline of the quadruped, a healthy example of the same species of quadruped, or as a differential between body parts of the quadruped.

The following embodiments and descriptions are for illustrative purposes only and are not intended to limit the scope of the System for Detecting Lameness in Sport Horses and other Quadrupeds. Other aspects and advantages of the present disclosure will become apparent from the following detailed description.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure. Also, the drawings included herein are considered by the applicant to be informal.

FIG. 1 is a block diagram in the physical layout of the Preferred Embodiment System Physical Layout.

FIG. 2 is a schematic diagram of the system in a preferred embodiment.

FIG. 3 is a block diagram indicating the components of the system in the preferred embodiment.

FIG. 4 is a sequence diagram indicating the typical system flow during operation.

FIG. 5 is a flowchart showing how data is processed.

FIG. 6 is a drawing indicating the Points of Interest (POI) on the horse.

FIG. 7 is a block diagram showing some of the processed data.

FIG. 8 is an example representation of a three dimensional point cloud of a horse.

FIG. 9 is a flowchart showing the alerting behavior of the system.

FIG. 10 is a flowchart showing data retrieval from the system.

FIG. 11 is a drawing of a quadruped foot showing angle.

FIG. 12 is a drawing of a quadruped leg showing fetlock angle.

### DETAILED DESCRIPTION

In the Summary above and in this Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, among others, are optionally present. For example, an article “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

### DEFINITIONS

Quadruped: An animal or species of animal having four legs and feet or hooves.

Locomotor: The muscular and skeletal system of the animal which governs movement.

Gait: The manner in which an animal or human walks or runs.

Symmetric Gait: A gait in which the left and right legs alternate equal movement. Humans do this naturally. With horses the only symmetric gaits are the walk and the trot, all other gaits are asymmetric.

Specimen: An individual animal or quadruped.

Computer Vision: A field that includes methods for acquiring, processing, analyzing, and understanding images and, in general, high-dimensional data from the real world in order to produce numerical or symbolic information, such as forms of decisions.

Lameness: An abnormal gait or stance of an animal that is the result of dysfunction of the locomotor system, typically caused by injury.

3-Dimensional (3D): Having representation in depth, width, and height dimensions.

3-Dimensional Representation: A form of representing the three dimensional position in space of a body or object.

Point Cloud: A series of points representing the position of objects in space. These points are usually defined by X, Y, Z coordinates, and often are intended to represent the external surface of an object. This is an example of a 3-Dimensional Representation.

Longitudinal Studies: Repeated studies of the same points over time, in order to establish a baseline and changes from that baseline over time.

Longitudinal Point Cloud: A series of collections of point cloud studies.

CDP: Computerized depth perception

Session: A period of time in which a specimen quadruped is visible to one or more of the camera collections.

Camera collection: A combination of camera, infrared camera, and computerized depth perception device all housed in the same physical unit. An example of this is the Microsoft Kinect.

Computer vision system: A camera collection.

API (Application Programming Interface): A set of interfaces or additional functionality provided by an external software library.

Open CV (Open Computer Vision): A popular and free open source computer vision set of APIs.

Haar Feature Based Cascade Classifier: A popular algorithm for object and feature recognition, available in the trademarked “OpenCV” API.

Machine Learning: A technique of computer science where a computer is taught to recognize or act based of a set of learned examples instead of being explicitly programmed.

Harris Corner Detection: A popular algorithm for detecting corners in an image, available in the trademarked “OpenCV” API.

Hough Lines Detection: A popular algorithm for detecting straight lines in an image, available in the trademarked “OpenCV” API.

Point of Interest: A point on the body of a quadruped calculated and tracked over time.

Mathematical representation of movement: Part of the processed data, indicating the positions, velocities, and accelerations of all points of interest as well as center of mass of a quadruped at a plurality of points in time.

Random Forest Decision Tree: A computer science machine learning technique.

A version of the disclosed invention comprises a system that utilizes at least one camera and one computerized depth perception apparatus (in total a camera collection) to automatically scan, capture and collect movement data from at least one physical object over time and a method of capturing, collecting, storing, analyzing, transmitting and displaying the data gathered without the need for attaching sensors to the body of the horse or quadruped, or requiring a force plates or expensive, high speed cameras is disclosed. This data is gathered and transmitted without the need for operator interaction.

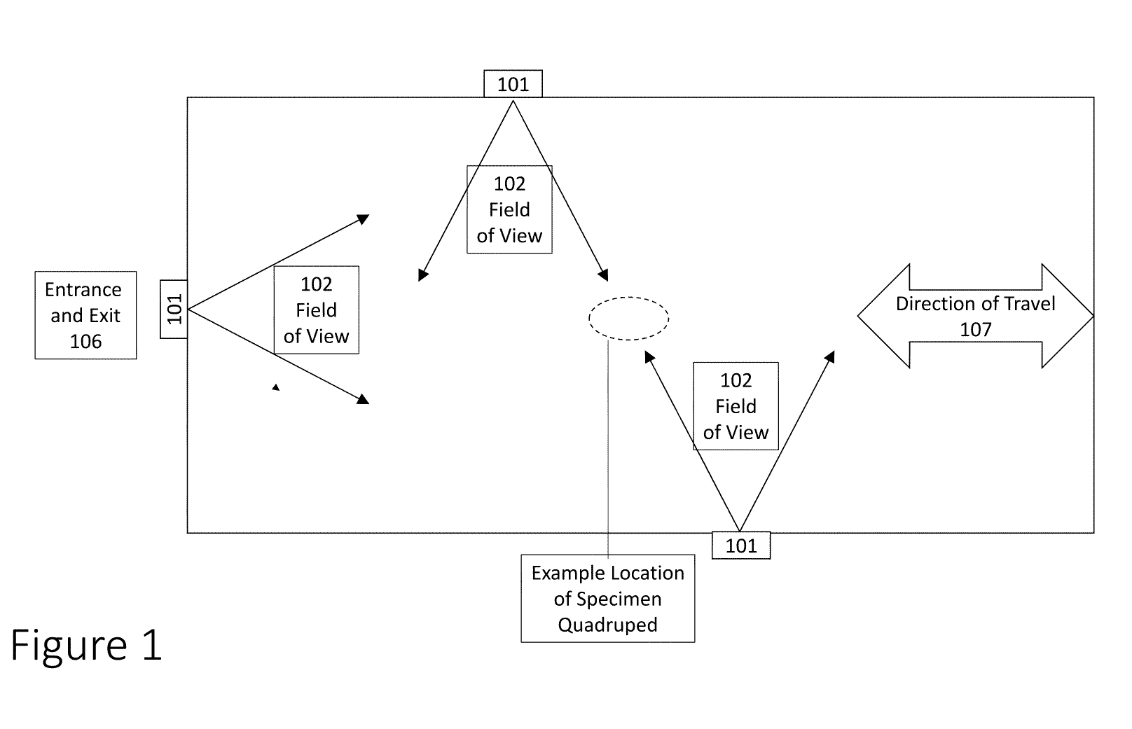

Specific details of certain embodiments are set forth in the following description and in FIGS. 1-12 to provide a thorough understanding of such embodiments. The present System for Detecting Lameness in Sport Horses and other Quadrupeds may have additional embodiments, may be practiced without one or more of the details described for any particular described embodiment, or may have any detail described for one particular embodiment practiced with any other detail described for another embodiment.

The system is comprised of one or more cameras that can capture still images and video in the human visible spectrum, one or more cameras that can capture still images and video in the infrared spectrum, and one or more CDPs that can record depth data over time. A combination of these cameras, or camera collections can be purchased today, such as Google's sensor, sold under the trademark “Project Tango” or Microsoft's sensor, sold under the trademark “Kinect”. In some embodiments, this camera collection is mounted in a barn (for example, a owner's or veterinarian's barn). In other embodiments, these camera collections are mounted on a portable apparatus on a veterinarian's body. In further embodiments, the camera collection could be mounted on a free moving remotely controlled aerial vehicle. None of this language is meant to limit the type or place of the mounting of the cameras. The cameras are connected to a computing device via a wired or wireless connection which runs the controlling and data processing software for the system. This computing device contains storage medium for storing the video, CPD data, and processed data as well as the capability to connect to the internet for transmission of said data. Some embodiments also contain a monitor or display to be able to feed back the calculations performed on the collected data

A preferred arrangement is shown in FIG. 1. The camera collections 101 are mounted in an area where the animal is likely to walk through, such as a horse barn or veterinarian barn. More specifically, the camera collection is mounted such that the horse direction of travel 107 is laterally in front of the cameras (left to right or right to left, as opposed to away and toward the camera collection). For embodiments with three or more camera collections, they are preferably mounted in series, alternating lateral sides of the walkway so that the continuous gait and movement of an animal can be captured. A final camera collection is placed oriented down the direction of travel, 105.

Another embodiment of the system is where a treadmill is used to get multiple strides of a specimen's movement. The camera collections are preferably mounted and oriented towards the treadmill. One camera collection is mounted to a wall or sturdy surface at a height of approximately at the middle vertical point on the animal being analyzed, at a lateral midpoint to the specimen, such that the specimen is perpendicular to the camera, presenting a profile view. All camera collections are mounted at such a distance that the full quadruped is in view of the camera collection. Another camera collection is mounted on the opposite lateral side. This placement differs from the placement of the camera collection in the preferred embodiment as the camera collections face each other. A third camera collection would be mounted facing the rear or caudal side of the specimen at a height midway up the body.

In other embodiments, the camera collection is not mounted and is instead free moving. These embodiments are considered portable camera collections in which each portable camera collection may be housed in a portable enclosure removably attached to the body of a system operator, or on a free moving automated device or vehicle, such as a drone. Additional embodiments consider camera collections from flying operators in the atmosphere above ground, in orbit or in outer space such as satellites or other aerospace vehicles.

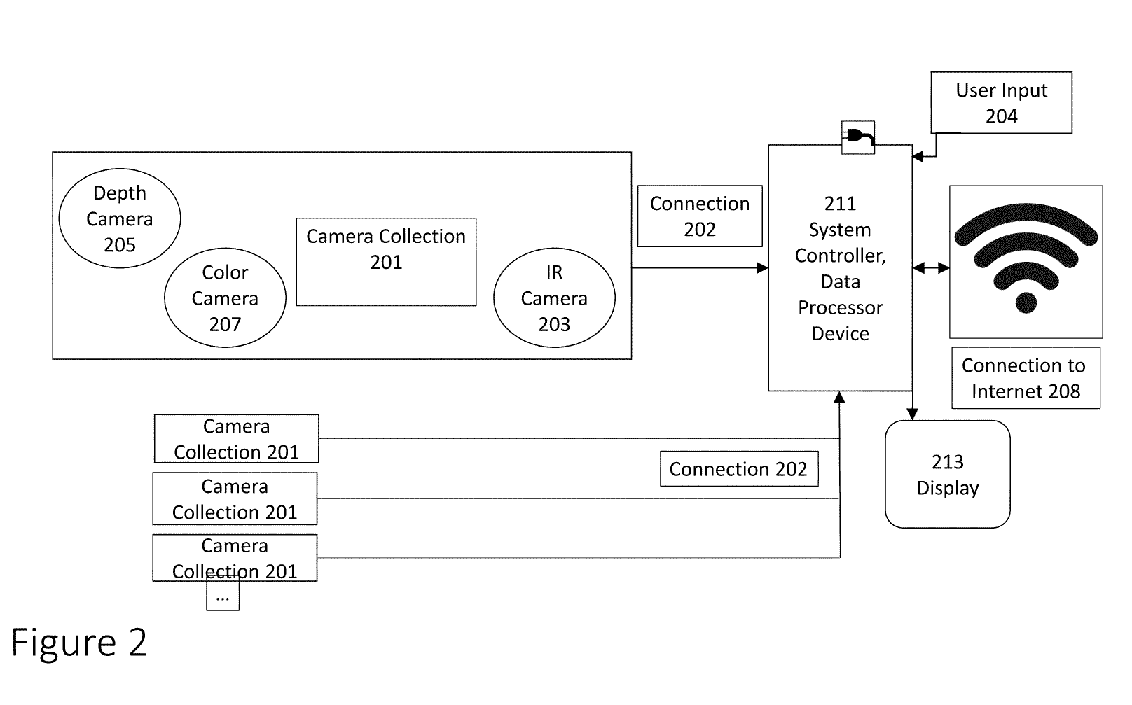

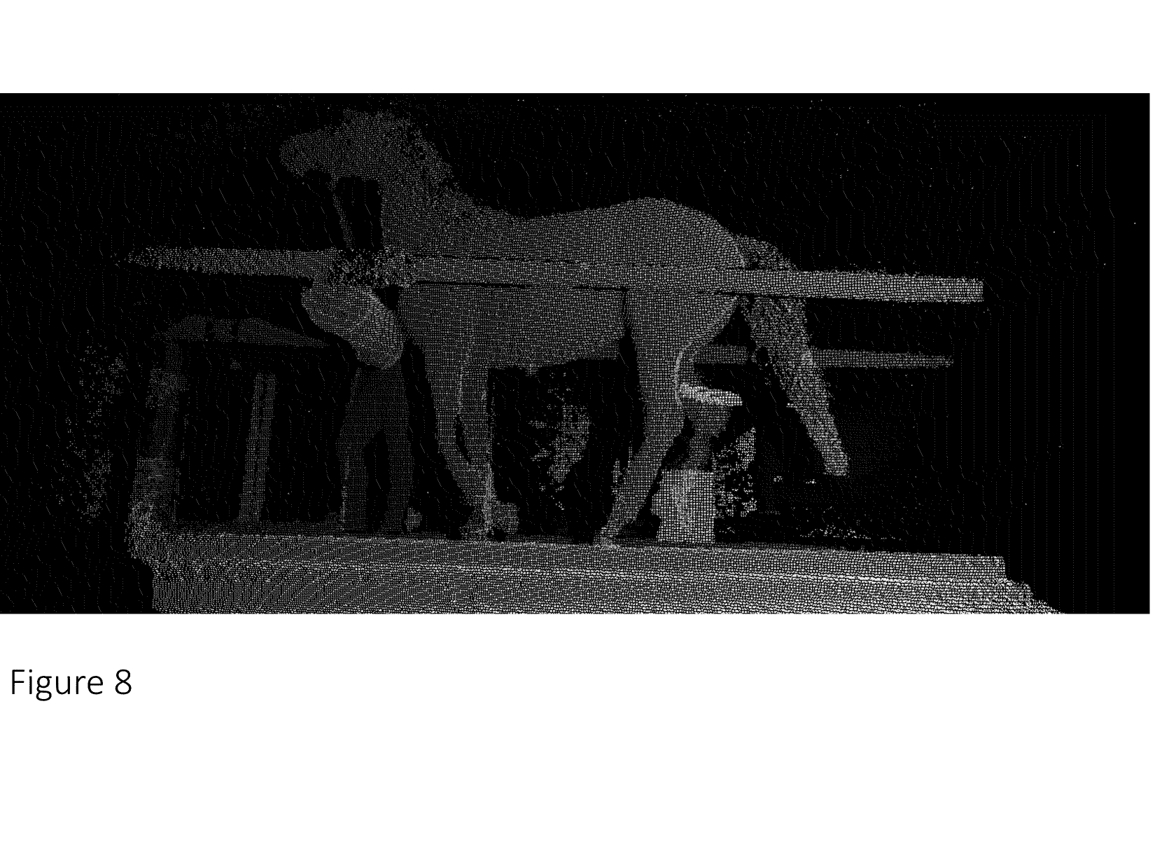

As shown in FIG. 2, a high-level schematic showing the preferred embodiment for a system configured to at least collect, transmit, control, process, store and display data collected by each camera collection in the system. The camera collection 201 preferably is a single container which comprises at least a depth camera 205, color camera 207 and an infrared camera 203. There may be multiple camera collections 209 connected via a wired or wireless connection 202 in series to the computer system 211. The computer system typically comprises a general-purpose computer processor, which is programmed in software to carry out the functions described herein below. The software may be downloaded to the processor in electronic form, over a network, for example, or it may alternatively be provided on non-transitory tangible media, such as optical, magnetic, or electronic memory media. Alternatively or additionally, some or all of the functions of the image processor may be implemented in dedicated hardware, such as a custom or semi-custom integrated circuit or a programmable digital signal processor (DSP). The three input signals of the infrared, the color, and the depth data are utilized by the system to create a point cloud representation of the movement of the animal. See FIG. 8. The computer system 211 is preferably connected to the internet and gathers inputs from the camera collection(s) and users (such as keyboards, mice, touch-commands, signals, and those common in the art) to output a display of data 213 to one or more displays as requested by the users. The computer system 211 is connected to the internet either wirelessly or wired via 208.

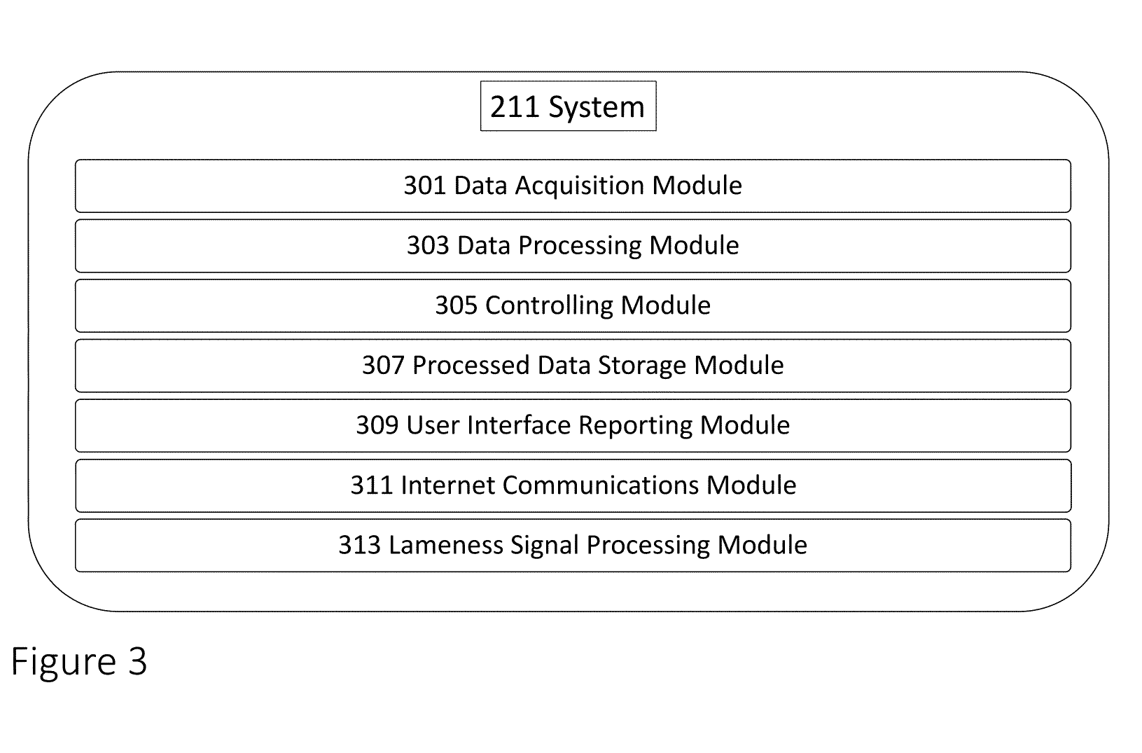

The computer system 211 is detailed in FIG. 3, showing the various modules within the computer system which perform varying functions to and with the data collected by the camera collection(s). A preferred set of module include at least a Data Acquisition module 301, the data processing module 303, the Controlling Module 305, The Processed Data Storage Module 307, the User Interface Reporting Module 309 and the Internet Communications Module 311. The interactions among the various modules are all managed by the computer system.

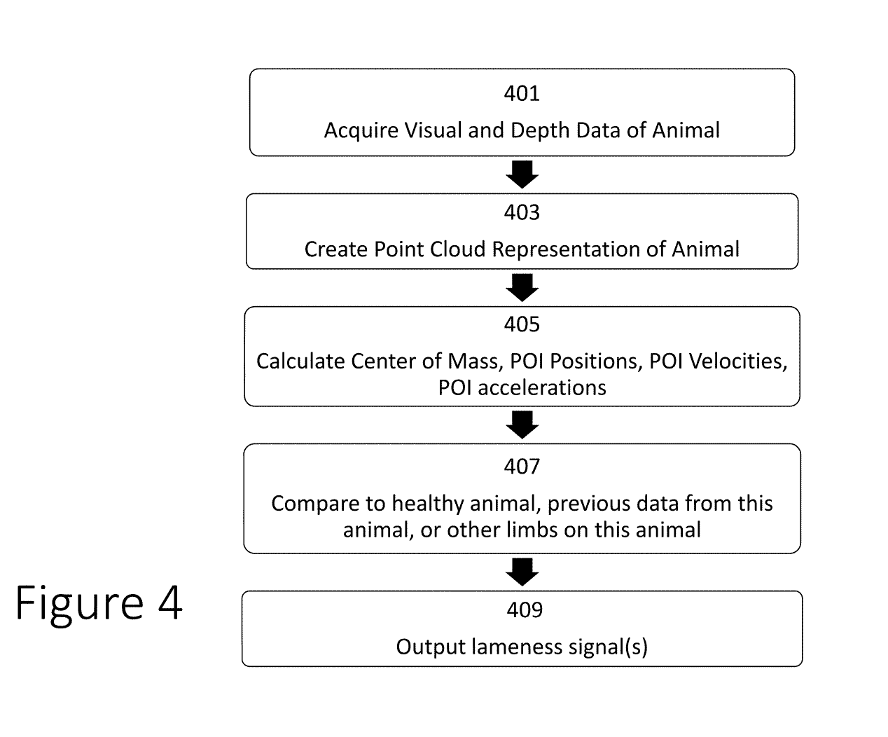

The Data Acquisition Module 301 generally performs the first step the flow chart shown in FIG. 4. The first step, acquisition 401 is to collect at least the three sources data (infrared, color and depth) from the specimen. Preferably, the acquisition data is collected while the animal is walking or trotting in front of the camera. The infrared signals collect and transmit a series of infrared images to the processor 303. The color signals provide a series of color images provided to the processor 303. The Depth signal provides a map of points with corresponding distances from the camera which is then transmitted to the processor 303. Each infrared image, color image, and depth image is transmitted to the processor along with a time stamp indicated at what time the images and map were captured.

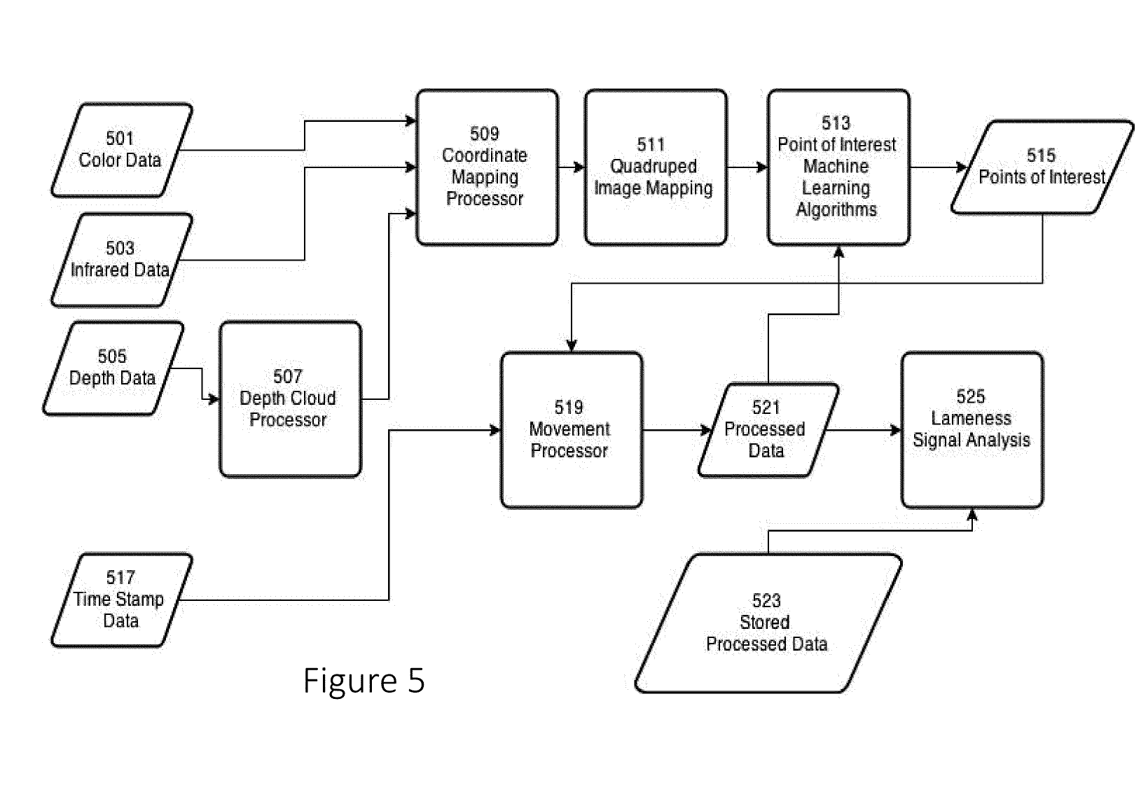

The Data Processing Module 303 processes data as described in FIG. 5. It takes the depth signal 505 and uses that to generate a point cloud 507. The point cloud is a 3D representation of where all points in the color and infrared images are in X, Y, and Z coordinate space. This point cloud is mapped onto the color and infrared images, utilizing the Microsoft Kinect 2.0 SDK CoordinateMapper functionality or other appropriate coordinate mapping functionality 509. Using the depth data and knowledge of the corresponding 3D space, all points in the color and infrared images that do not pertain to the quadruped specimen are eliminated 511. The cleaned images containing only the color and infrared images pertaining to the quadruped then undergo a series of steps to identify points of interest. The data is passed to 513 the point of interest trained machine learning algorithms. As additional input, the processed data 521 from previous iterations in time is included to help with error smoothing. These machine learning algorithms include, but are not limited to, a Haar based cascading classifier and a random forest decision tree. Machine learning data from previous movement of the same species of animal to determine joint positions and other points of interest. The Haar based cascading classifier utilized is an API available in open software repositories such as one by OpenCV, trademarked as “OpenCV”. Other embodiments could use any machine learning classification system. The X, Y, and Z coordinates of the POI data 515 is then combined with the timestamp data 517 and passed to the movement processor 519 which calculates the velocity and acceleration of the POI at each point in time, known as the processed data 521. The processed data is then used to make a lameness determination 525 along with prior processed data from previous sessions of the gait analysis system 523 with the quadruped specimen.

The Controlling Module 305 is detailed in FIG. 4. The controlling module communicates with each component of the system, directing the flow through each step of the process. 401 The controlling module uses the data acquisition component 301 to obtain color images, infrared images, and depth data from each camera collection. This data is passed to the data processing component 303 to obtain one or more lameness signals 405. The controlling module further directs the data processing component to process data from previous sessions that the controlling module has loaded from the data storage module 307.

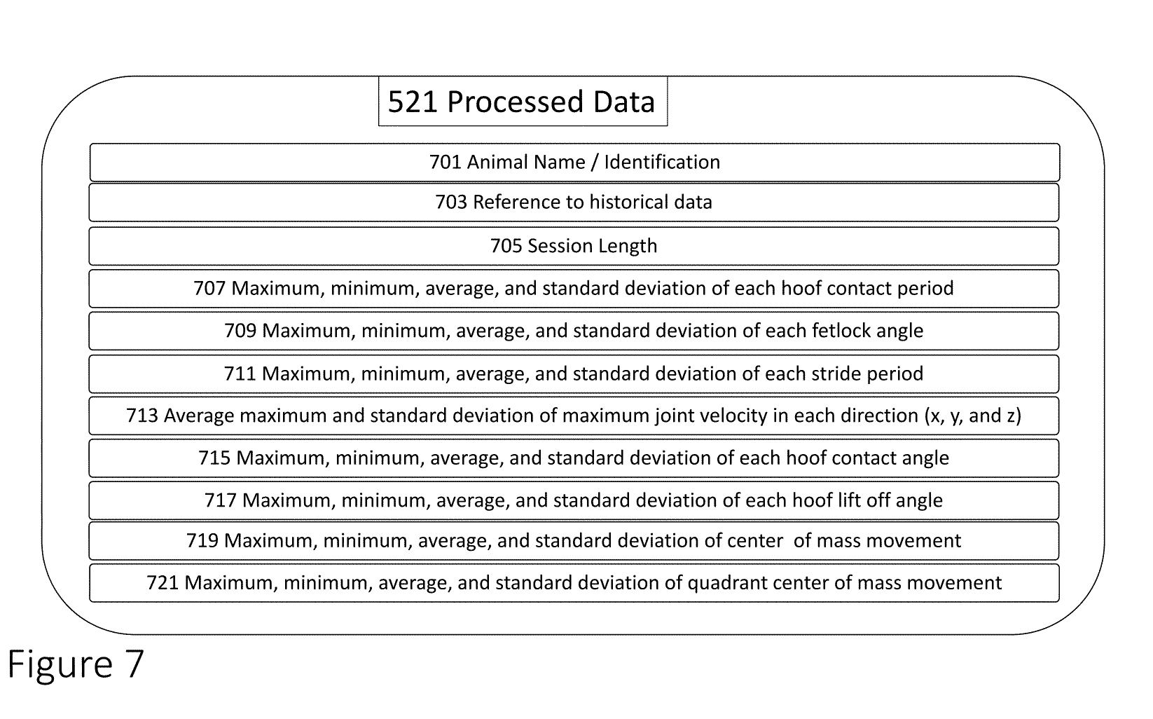

The Processed Data Storage Module 307 receives processed data from the controlling module and stores this in a permanent record in database format. It also responds to data requests from the controlling module for historical or baseline processed data. In some embodiments storing permanently in memory the processed data for each quadruped from FIG. 7, the system creates a baseline of an individual specimen. The data is recorded from each instance the system observes a specimen. This processed data is stored in a database file format known to the art on the data processing and control machine. When the specimen is present for further evaluations by the system, the system will measure and report deviations from the specimen's calculated baseline, as well as deviations between distal limbs. For example, the specimen may be a sport horse and a baseline may be created at a time when the sport horse is known to be healthy. By using this system to monitor the particular sport horse continuously for even slight deviations from the baseline, the system detects even subtle deviations in gait, or other posture or movement characteristic detailed in the processed data (see FIG. 7), to alert caretakers before any potential injury becomes exacerbated by continued exertion and/or lack of attention and treatment.

In some embodiments, the Data Processing Module 303 within the Computer System is configured to capture and retain additional data upon the detection of a predetermined event, such as a deviation from an individual specimen's healthy baseline gait. For example, the system may be configured to automatically begin to record video, 3D CAD video representation when there is abnormal data collected of a horse in addition to the normal processed data. This additional data is store in the Data Storage module 307.

The User Interface Reporting Module 309 allows a third party such as the operator, user, or owner of the specimen to control the system by adding information about specimens, requesting previous data, requesting current data of a specimen under examination, or configuring thresholds for changes in lameness signals.

The Internet Communications Module 311 manages communication between the system and other systems via medium of the internet. It is responsible for transmitting at least the alert and data of changes when instructed by the controlling module 305.

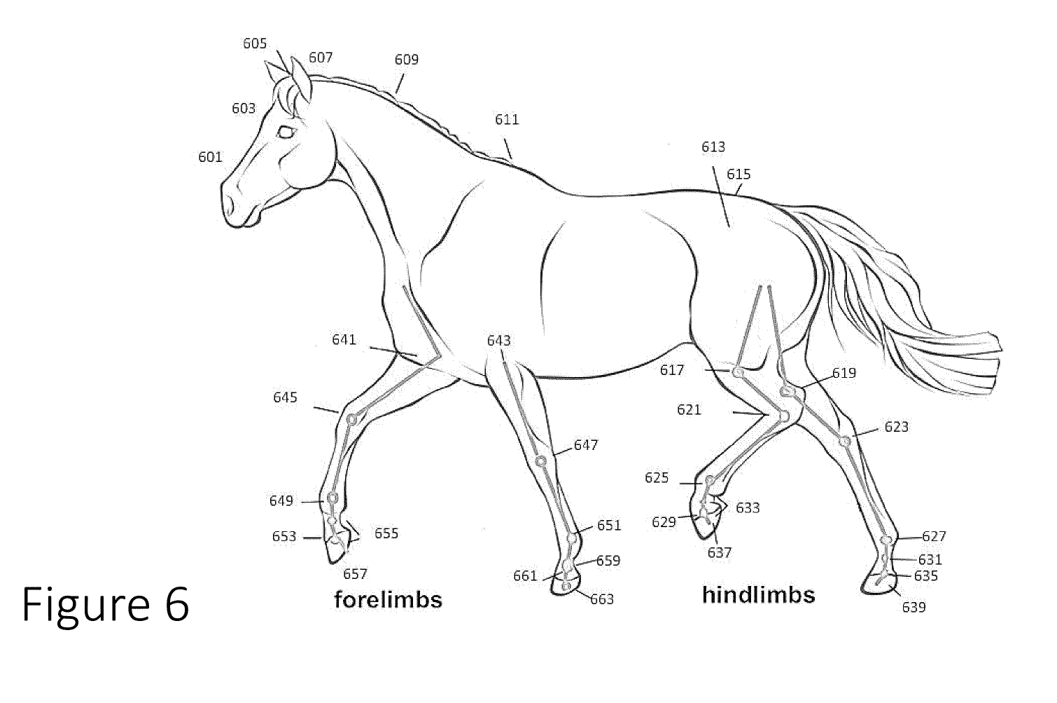

FIG. 6 shows some of the points of interest (POI) on an example quadruped. The list is illustrative to show what might be captured as a POI, but should not be construed to be exhaustive or complete, other POIs could be recorded. In order to analyze a horse for example, at least the following positions would need to be captured: muzzle 601, eyes 603, poll 605, ears 607, crest 609, withers 611, tuber coxae 613, sacrum 615, left stifle 617, right stifle 619, left hock 621, right hock 623, left hind fetlock 625, right hind fetlock 627, left hind interphalangeal joints 629 and 633, right hind interphalangeal joints 631 and 635, left hind limb hoof 637, right hind limb hoof 639, right forelimb elbow 641, left forelimb elbow 643, right carpus 645, left carpus 647, right forelimb fetlock 649, left forelimb fetlock 651, right forelimb interphalangeal joints 653 and 655, left forelimb interphalangeal joints 659 and 661, right forelimb hoof 657, left forelimb hoof 663.

Using the POIs gathered from the previous step, as well as joint positional data from previous frames in the same session, changes in movement such as velocity and acceleration of the POIs is calculated and recorded at that moment in time. Since the positions are in 3D space, these velocities and accelerations will be in each of the coordinate X, Y, and Z directions. This becomes part of the processed data. See FIG. 7.

FIG. 7 shows processed data 521 that is stored in the Processed Data Storage Module 307 comprised of: 701 Animal Name/Identification, which is a unique reference to a specimen that is being examined, 703 Reference to historical data, which is a means to look up previous data for the specimen or the specimen's species, 705 Session Length, the length in time of a session measured in seconds, 707 maximum, minimum, average, and standard deviation of each hoof contact period as measured in seconds, 709 maximum, minimum, average, and standard deviation of each fetlock angle when each foot is at rest during the session measured in degrees, 711 maximum, minimum, average, and standard deviation of each stride period measured in seconds, 713 average maximum and standard deviation of maximum joint velocity and acceleration for each POI in each direction (x, y, and z), measured in meters per second, 715 maximum, minimum, average, and standard deviation of each hoof contact angle measured in degrees, 717 maximum, minimum, average, and standard deviation of each hoof lift off angle, and 719 the maximum, minimum, average, and standard deviation of the center of mass movement in each direction (x, y, and z), 721 the maximum, minimum, average, and standard deviation of quadrant center of mass movements and accelerations.

In some embodiments using the point clouds, a volume and weight distribution of a quadruped can be determined by making a constant density assumption of the animal and calculating based off volume. Volume and center of mass are calculated at the same time. The volume can be determined by calculating the X, Y space occupied by an individual point in the depth map, then multiplying this by the depth in the Z direction from the depth points. V_{XY}=dx×dY×depth. The summation of all points X and Y in the depth cloud space in the quadruped's body and the volumes at each point sum to the total volume. V=ΣV_{XY} The center of mass is then determined by making a constant density assumption of the body of the quadruped, Center of

$${Mass}_{x} = \frac{\sum {V}_{XY} \times X}{V}$$

where V_{xy} is the volume at position X, Y, and X is the position in the X direction, and V is the previously calculated volume.

The processed positional data is then measured over the time period of the session. For each of these POI positional measurements, a maximum acceleration and maximum velocity is be determined for each point separately. A standard deviation of that data is recorded. The positional data is then used to calculate frequency of movement (as joints accelerate ad decelerate) to determine stride duration. The length of time that each hoof is in contact with the ground is also recorded, as well as an average length of time over multiple steps as well as the standard deviation.

In some embodiments, once the system has captured and processed this data, analysis is performed on the data to determine the presence and severity of lameness (lameness signals). This analysis is done via several methods and agreement between these signals is utilized to determine certainty of lameness. The following listed methods should not be construed to be exhaustive or complete, other lameness signals known to the art could be utilized.

One method of data analysis measures the 3D movement around the hips, especially the gluteal muscles, sacrum and tuber coxae. Studies have shown that horses will elevate and drop their hip asymmetrically to compensate for pain in a hind limb lameness. The maximum, minimum, average, and standard deviation of the positions, velocities, and acceleration of dorsal points of the hip on both sides (tuber coxae covered by gluteal muscles) in all 3 coordinate planes, as well as which hooves are moving as part of the processed data. Differential in the tuber coxae movement between lateral sides of the quadruped is a lameness signal, where a greater differential indicates greater lameness.

Another method of data analysis measures head bob, which is the movement of the head in comparison with the stride of the legs. Studies' have shown that horses will utilize their heads to offset the weight that comes down on their legs during different parts of the stride. The maximum, minimum, average, and standard deviation of Y (or upward and downward) movement of the quadruped specimen's head is recorded, as well as which feet are moving and which still (stride data) form part of the processed data. Movement of the head in the Y direction is a lameness signal, where greater movement during a phase of the trot than another indicates lameness, the greater differential indicating greater lameness.

Another method of data analysis to measure the relative maximum velocities and accelerations of the hooves and all other points of interest. Research shows a differential between hooves' maximum velocities when one leg of a horse is lame. These velocities and accelerations are measured in each of the X, Y, and Z dimensions. Velocity is measured by measuring the change in position of a point of interest or hoof between individual images and measuring the time elapsed between those. V_{X POI}=(X₂−X₁)×(T₂−T₁) where T₁ and T₂ are times at individual image times. X₁ and X₂ represent positions in the X plane at those times. The acceleration of those POI's is calculated A_{X POI}=(V₂−V₁)×(T₂−T₁) where T₁ and T₂ are times at individual image times. V₁ and V₂ represent velocities in the X plane at those times. The maximum and minimum is recorded over the duration of a single stride. The average of each of those over multiple strides is then calculated, as well as the standard deviation. The differential between these values between points of interest on each side of the quadruped, as well as the differential between these values and the specimen's sound baseline data, as well the differential between these values and the baseline data for the specimen species constitutes a signal of lameness, where the greater the difference indicates greater lameness.

Another method of data analysis measures location where each hoof is placed in relation to the quadruped's path of movement and to the other hooves. Studies have shown that in some lameness the horse may drift away from the lame limb, so that the lamb limb tracks under the body. The location of each hoof, including maximum, minimum and standard deviation of distance of each hoof in relation to the quadruped's center of mass is used to determine lameness of an individual limb, where the greater the differential of the position of the hooves compared to the center of mass indicates lameness in that limb.

Another method of data analysis measures stance, which is the symmetry in the horse as it stands still, in a resting position. Some variations in muscle mass, joint flexion, and hoof (angle, wear, shape) may vary with chronic lameness. Measurements of these will be made, including angles and shape of hoof, and all points of interest as well as joint angles will be recorded, as well as asymmetries between sides. Asymmetries in the sides is used as a lameness signal, where larger asymmetries indicate greater lameness.

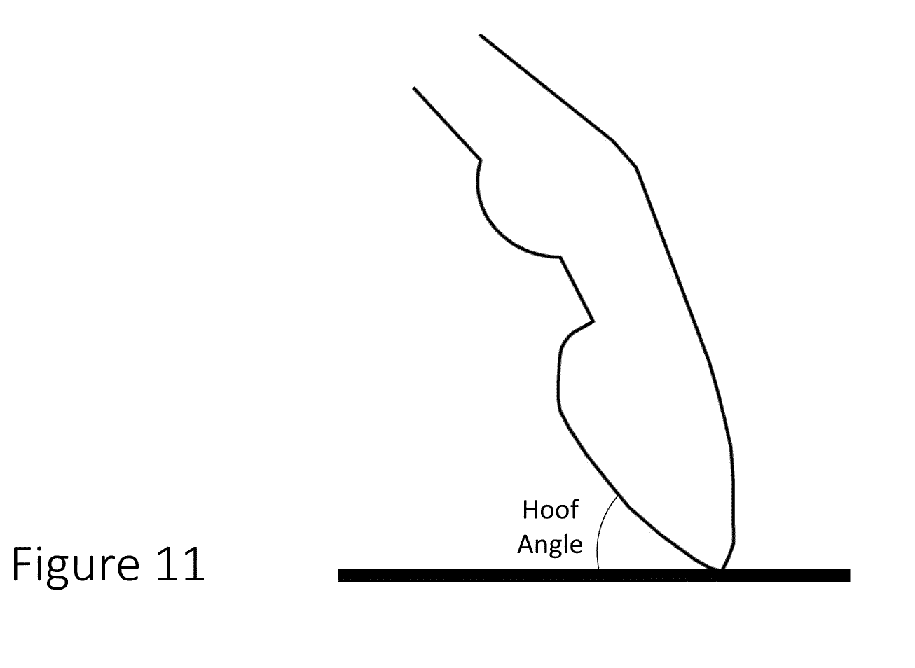

Another method of data analysis is a measure of the angle of a hoof to the floor as well as sagittal plane. See FIG. 11. This angle is measured at different points of the movement of the leg, such as at the moment when the toe is lifted off from the ground and when the hoof strikes the ground again. Forelimbs of horses have a smaller angle of their hooves to the sagittal plane (i.e. closer to the sagittal plane) (research reference). If a differential exists between sides of the horse a signal of lameness is determined based on the severity of the differential.

Another method of data analysis utilizes the time of contact differential between the hooves on each side of the quadruped. The processed data contains a value for the average time of contact of each hoof during each step of the specimen's gait. Measuring the difference between each side of the quadruped constitutes another signal of lameness.

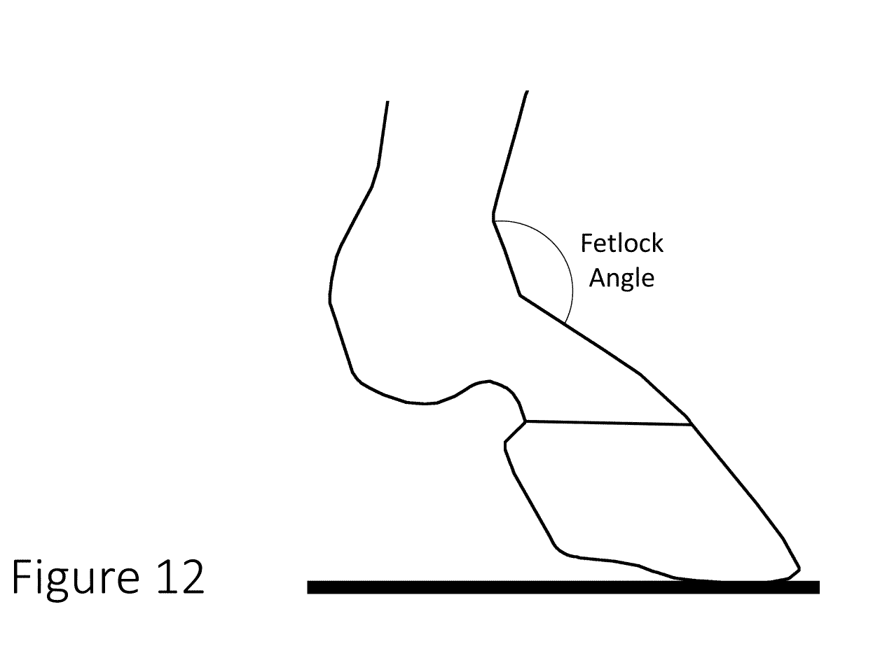

Another method of data analysis measures the fetlock angle of the quadruped when that leg is in contact with the ground (see FIG. 12). Studies have shown that the fetlock angle of a horse is proportional to peak vertical force that is put on an individual leg. As a result, lame legs will often have a shallower fetlock angle when in contact with the ground. The measurement of each fetlock angle at each step is part of the processed data, and the differential between fetlock angles during periods of hoof contact is a lameness signal, where a larger differential represents a larger lameness signal.

Another method of data analysis would measure differential between the normal movement of the center of gravity or center of mass for the quadruped and the measured movement. This measurement occurs in all three dimensional planes. This measurement measures the distance traveled, maximum acceleration, and maximum velocity in each direction of the center of mass.

Another method of data analysis utilizes machine learning. All of the processed data for the quadruped is processed by a machine learning algorithm. This machine learning algorithm has been trained with recorded data against a plurality of horses and other quadrupeds in various states of lameness and soundness of health. The machine learning algorithm then stores with the processed data an indicator of diagnosis of both lameness and severity. The machine learning algorithms could utilize a cascading classifier, supervised machine learning algorithms such as artificial neural networks, random forest decision trees, Bayesian networks, hidden Markov models, as well as others known to the art trained on a plurality of quadrupeds in various states of lameness and soundness of health.

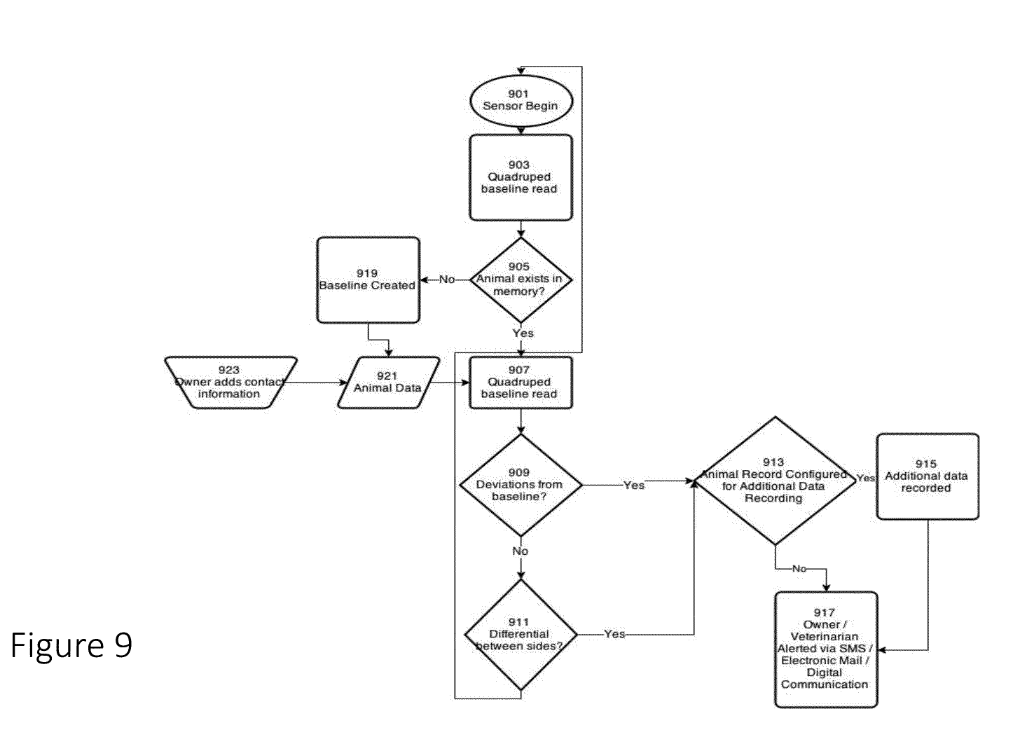

FIG. 9 describes an optional alerting feature of the Computer System. The alerting feature is configured to alert one or more predetermined interested parties upon the detection of a predetermined event, such as a deviation from an individual specimen's healthy baseline gait. At 901, the sensor is actively looking for a specimen. At 903, a specimen is capture by one or more of the camera collections. At 905, a machine learning algorithm is utilized to determine if the animal was previously known. If the specimen was not, the data from the session becomes a baseline for the specimen at 919. The owner of the animal has the option of entering data at any time in order to register the specimen with the system at 923. This data entry would include configurable thresholds for changes in lameness signals. If the animal does exist in the database of processed data, the baseline data for the individual animal is loaded for comparison at 907. At 909, a determination if there is a deviation that meets the programmed threshold from the baseline is made. At 911, a determination if there is a deviation between lateral sides of the quadruped is made that meets the programmed threshold from the baseline. If such a deviation does exist, additional information from the session such as video and entire plurality of point clouds is recorded in the data storage module. The system may be configured to automatically transmit electronic alerts 917, which can be done via electronic mail, SMS (text messaging to phones), or other electronic notification in another copy of the lameness system. This alert can include generated 3D CAD video representations of particular specimens as well as video of the specimen upon a presumed deviation in gait. The alerted parties may then take appropriate action. First, the computer system defines a baseline for a subject quadruped and by using the processed data storage module 307

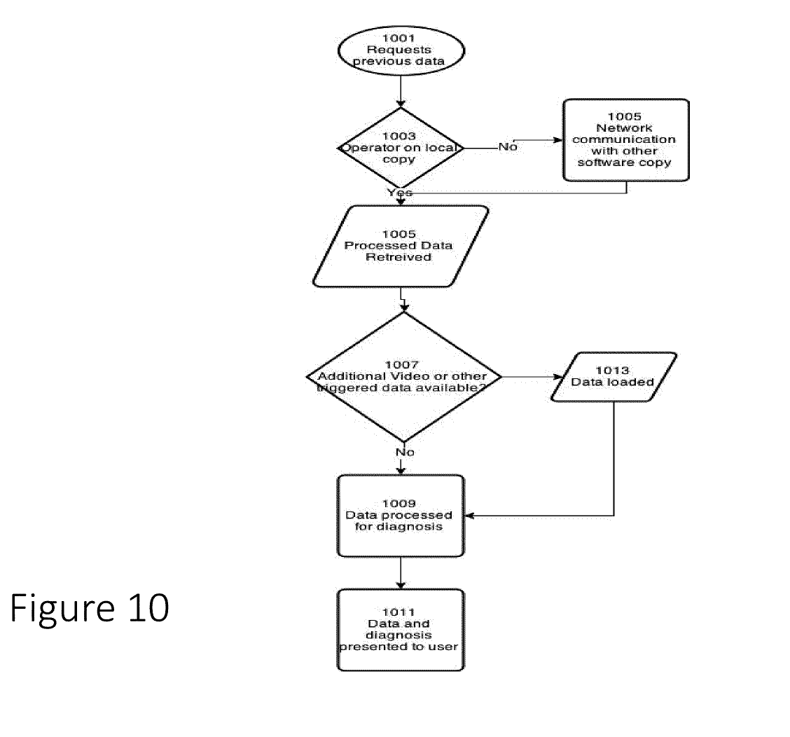

In some embodiments, the System for Detecting Lameness in Sport Horses and other Quadrupeds can be used to retrieve information about a specified specimen. (See FIG. 10.). By utilizing the input keyboard and mouse on the local System, an operator can retrieve the stored processed data 1001 and diagnosis 1009. The operator can also utilize a remote System of the same software to communicate to other networked Systems to retrieve data 1013. By retrieving this data, the operator is shown plain text differentials of all processed data (FIG. 7), as well as lameness signal. If a plurality of lameness signals has been determined, and the system was configured to store additional data upon a diagnosis of lameness 1007, the operator can play back video and 3D CAD video representations of the specimen's movement as well. This data is presented to the operator at step 1011.

In some embodiments, the system is configured to permanently store all data collected on designated specimens. Furthermore, in preferred embodiments the system individually identifies specimens thereby eliminating the need for manual input of specimen identification by caretakers. For example, in some embodiments the system identifies particular specimens through comparison of current data input, such as real time computer vision depth perception sensory input, with previously collected and/or uploaded data associated with the same specimen. In some embodiments, a Haar based cascading classifier is utilized to identify an individual animal. In other embodiments, the system identifies particular specimens through use of Radio-frequency identification (RFID), such as an RFID tag, may be attached to or implanted within each individual specimen. Any other suitable type of identification method known in the art may also be used without departing from the spirit and scope of the System for Detecting Lameness in Sport Horses and other Quadrupeds.

In some embodiments, the system enables individual animal owners, veterinarians, and/or other caretakers to retrieve information about the animal. This information is then displayed in a way that allows either a point in time or longitudinal view of the data stored in the system. This retrieval and display of the accumulated data, analysis, and diagnosis can be used to better understand the history and current state of a horse.

While preferred and alternate embodiments have been illustrated and described, as noted above, many changes can be made without departing from the spirit and scope of the System for Detecting Lameness in Sport Horses and other Quadrupeds. Accordingly, the scope of the System for Detecting Lameness in Sport Horses and other Quadrupeds is not limited by the disclosure of these preferred and alternate embodiments. Instead, the scope of the System for Detecting Lameness in Sport Horses and other Quadrupeds should be determined entirely by reference to the claims. Insofar as the description above and the accompanying drawings (if any) disclose any additional subject matter that is not within the scope of the claims below, the inventions are not dedicated to the public and Applicant hereby reserves the right to file one or more applications to claim such additional inventions.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example of a generic series of equivalent or similar features.

Any element in a claim that does not explicitly state “means for” performing a specified function, or “step for” performing a specific function is not to be interpreted as a “means” or “step” clause as specified in 35. U.S.C. §112 ¶6. In particular, the use of “step of” in the claims herein is not intended to invoke the provisions of U.S.C. §112 ¶6.

What is claimed is:

1. A gait detection apparatus, comprising:
a computer vision system operable to perceive the depth of a plurality of points on a specimen quadruped;
a control system being operable to at least:
receive an input from the computer vision system;
generate from the input a plurality of three-dimensional representations of the specimen quadruped, wherein at least two of the plurality of three-dimensional representations correspond to a different time from each other;
utilize the plurality of three-dimensional representations for the creation of a mathematical representation of a movement of the specimen quadruped;
update the mathematical representation of a movement of the specimen quadruped and detect deviations in the representation of the movement over time.

2. A method of determining lameness in a quadruped comprising:
One or more camera collections that captures image data of at least infrared, color and depth of a specimen quadruped;
A first computer system that creates data points in three dimensional space from the image data to produce a point cloud, recognizes specific three dimensional points of interest from the point cloud that are associated with points of interest on the specimen quadruped, collect motion data of at least position, velocity and acceleration data of each point of interest over time;
A second computer system that generates one or more lameness signals by comparing a set of processed motion data of the specimen quadruped based on the motion of points of interest to that of a baseline set of motion data for a generic species of the specimen quadruped, comparing the processed motion data of the specimen quadruped to a baseline set of processed motion data of the specific specimen, and comparing the processed motion data of the specimen quadruped with other processed motion data of the specimen quadruped; and
A third computer system that transmits the level of severity of one or more lameness signals of the specimen quadruped.

3. The method of claim 2 wherein a first camera collection is positioned facing the direction of travel of a specimen quadruped and a second camera collection is positioned facing the lateral side of the specimen quadruped, perpendicular to the first camera collection.

4. The method of claim 3 wherein a third camera collection is positioned opposite the second camera collection, facing the opposite lateral side of the specimen quadruped in a staggered positioned such that the second and third facing camera collections' field of view is approximately continuous such that a continuous field of view of the second camera collection to the third camera collection is created.

5. The method of claim 2 wherein a quadruped's head is a point of interest, head movement is the processed motion data and the greater the head movement the greater the lameness signal.

6. The method of claim 2 wherein a quadruped's fetlock joints are points of interest, fetlock joint angle motion over time is the processed motion data and the greater the differential in fetlock joint angle the greater the lameness signal.

7. The method of claim 2 wherein a quadruped's hooves are points of interest, hoof motion is the processed motion data and the greater the difference between hoof processed motion data the greater the lameness signal.

8. The method of claim 2 wherein a quadruped's hooves are points of interest, hoof position relative to the center of gravity of the quadruped are the processed motion data and the greater the differential of hoof position relative to center of gravity of the quadruped the greater the lameness signal.

9. The method of claim 2 wherein a quadruped's collective processed motion data is further processed by a machine learning algorithm.

10. The method of claim 2 wherein a quadruped's hooves are a points of interest and the quadruped's relative hoof angle to the ground level are the processed motion data and the greater the differential of hoof angle the greater the lameness signal.

11. The method of claim 2 wherein a quadruped's dorsal points are points of interest and hip movement is the processed motion data and the greater the asymmetry of the hip movement the greater the lameness signal.

12. The method of claim 2 wherein a quadruped's stance is derived from a plurality of points of interest of the specimen quadruped and asymmetry in stance is the processed motion data and the greater the asymmetry, the greater the lameness signal.

13. The method of claim 4 wherein at least one of the camera collections is removably attached to a mobile operator positioned near the specimen quadruped.

14. The method of claim 2 wherein the third computer system is further configured to transmit to one or more users when one or more lameness signals increases or decreases beyond one or more specified thresholds.

15. The method of claim 2 wherein the second computer system is further configured to provide one or more users the level of severity of one or more lameness signals.

16. The method of claim 2 wherein the second computer system is further configured to recognize unique specimen quadrupeds.

17. The method of claim 2 wherein the computer system recognizes one or more unique specimen quadrupeds by using a trained machine learning algorithm.

18. The method of claim 3 wherein one or more camera collections are added in a staggered positioned such that the subsequent camera collections' field of view is approximately continuous such that a continuous field of view from the prior camera collection to the added camera collection is created.

19. The method of claim 3 wherein at least one of the camera collections is removably attached to an aerospace vehicle.

20. The method of claim 3 wherein at least one of the camera collections is removably attached to a land vehicle.
